(19) United States
(12) Patent Application Publication
(10) Pub. No.: US 2023/0307133 A1
(43) Pub. Date: Sep. 28, 2023

(54) METHOD AND SYSTEM FOR GENERATING A PATIENT-SPECIFIC CLINICAL META-PATHWAY USING MACHINE LEARNING

(71) Applicant: Quai.MD, Ltd., Neve Ilan (IL)

(72) Inventors: Chen SHAPIRA, Haifa (IL); Golan YONA, Tel Aviv (IL); Shlomi UZIEL, Neve IIan (IL)

(73) Assignee: Quai.MD, Ltd., Neve Ilan (IL)

(21) Appl. No.: 18/189,634

(22) Filed: Mar. 24, 2023

Related U.S. Application Data
(60) Provisional application No. 63/269,859, filed on Mar. 24, 2022.

(51) Int. Cl. G16H 50/20; G16H 50/70

(52) U.S. Cl. CPC G16H 50/20; G16H 50/70

(57) ABSTRACT

A system and method for generating a patient-specific clinical meta-pathway. The method includes constructing a clinical meta-pathway graph of a network of pathway states, wherein each pathway state is associated with at least one of a plurality of differential diagnoses of a chief complaint; and wherein each pathway state includes a set of rules that define a connecting pathway state; applying a trained model to the constructed clinical meta-pathway graph to update the set of rules that define the connecting pathway state, wherein the model is trained based on at least historical patient data; navigating through the clinical meta-pathway based on input patient data, wherein the navigation through pathway states is guided by a function of the set of rules; and causing a display of at least a portion of the navigating pathway states.

### CROSS-REFERENCE TO RELATED APPLICATIONS

This application claims the benefit of U.S. Provisional Application No. 63/269,859 filed on Mar. 24, 2022, the contents of which are hereby incorporated by reference.

### TECHNICAL FIELD

The present disclosure generally relates to clinical pathways and more particularly to machine learning techniques to generate an interactive network of clinical pathways.

### BACKGROUND

Clinical pathway is a tool to guide evidence-based healthcare for clinical problems with a goal of providing organized and standardized healthcare processes. Other common terms for such clinical pathways may be clinical protocols or best-practices, but for the sake of generality, all such step-by-step clinical guidance may be referred to as clinical pathways. Such clinical pathway facilitates the decision-making process for physicians by providing a step-by-step guidance to improve patient safety and clinical efficiency. To this end, adherence to such pathways provides advantages of improving patient outcomes, reducing variations in diagnoses, costs, and more.

However, currently developed clinical pathways are isolated protocols that often focus on a single diagnosis or disease group. Such isolation and limitation do not reflect the complex, dynamic interconnections of diseases and the human body, and can cause inaccurate decisions. As an example, chest pain is a common complaint associated with more than 50 diseases where some are more frequent and easier to diagnose, but others are rare and may be more complicated to diagnose. Using the currently developed clinical pathway, many diagnoses related to chest pain that are outside the scope of a selected isolated protocol can be unintentionally omitted and undiscovered to risk patient wellness.

Moreover, it has been identified that the current system of clinical pathways takes a considerable amount of time to be developed and further implemented in clinical settings. Contents of the clinical pathways require extensive research for accurate representation of relevant components such as tests, treatments, communications, and more. Once established, such carefully developed clinical pathways are often distributed in document files or in siloed applications with the expectation for physicians to abide to them. Here, the clinical pathways are rather stagnant due to effort, time, and resources required to develop. And thus, modification of clinical pathways to reflect latest clinical results or to adapt to particular communities or facilities is challenging and slow.

Current evidence-based pathways are also limited in their fidelity as only a small fraction of patient parameters, based on an “average patient,” are considered and the large variability between patients and associated patient parameters are left out in constructing the clinical pathways. Moreover, decisions within the clinical pathway are often semi-rigorous decisions (e.g., hard thresholds, utilizing a limited set of variables and/or tests) derived from limited clinical studies conducted on relatively smaller groups of patients, typically within a certain geographical region. To this end, such current clinical pathways do not appropriately represent the diverse population and thus, ill-suited for applying to all patient cases.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for generating a patient-specific clinical meta-pathway. The method comprises: constructing a clinical meta-pathway graph of a network of pathway states, wherein each pathway state is associated with at least one of a plurality of differential diagnoses of a chief complaint; and wherein each pathway state includes a set of rules that define a connecting pathway state; applying a trained model to the constructed clinical meta-pathway graph to update the set of rules that define the connecting pathway state, wherein the model is trained based on at least historical patient data; navigating through the clinical meta-pathway based on input patient data, wherein the navigation through pathway states is guided by a function of the set of rules; and causing a display of at least a portion of the navigating pathway states.

Certain embodiments disclosed herein also include a non-transitory computer readable medium having stored thereon causing a processing circuitry to execute a process, the process comprising: constructing a clinical meta-pathway graph of a network of pathway states, wherein each pathway state is associated with at least one of a plurality of differential diagnoses of a chief complaint; and wherein each pathway state includes a set of rules that define a connecting pathway state; applying a trained model to the constructed clinical meta-pathway graph to update the set of rules that define the connecting pathway state, wherein the model is trained based on at least historical patient data; navigating through the clinical meta-pathway based on input patient data, wherein the navigation through pathway states is guided by a function of the set of rules; and causing a display of at least a portion of the navigating pathway states.

Certain embodiments disclosed herein also include a system for generating a patient-specific clinical meta-pathway. The system comprises: a processing circuitry; and a memory, the memory containing instructions that, when executed by the processing circuitry, configure the system to: construct a clinical meta-pathway graph of a network of pathway states, wherein each pathway state is associated with at least one of a plurality of differential diagnoses of a chief complaint; and wherein each pathway state includes a set of rules that define a connecting pathway state; apply a trained model to the constructed clinical meta-pathway graph to update the set of rules that define the connecting pathway state, wherein the model is trained based on at least historical patient data; navigate through the clinical meta-pathway based on input patient data, wherein the navigation through pathway states is guided by a function of the set of rules; and cause a display of at least a portion of the navigating pathway states.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is an example network diagram utilized to describe the various embodiments.

FIG. 2 is a flowchart illustrating a method for generating an interactive clinical meta-pathway according to an embodiment.

FIG. 3 is a flowchart illustrating a method for constructing a clinical meta-pathway graph by generating pathway states according to an embodiment.

FIG. 4 is a schematic diagram of a simplified clinical meta-pathway according to an example embodiment.

FIG. 5 is an example of a graphical user interface for interacting with the clinical meta-pathway according to an example embodiment.

FIG. 6 is a schematic diagram of an analysis system according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various disclosed embodiments provide a system and method for generating an interactive clinical meta-pathway that includes an interconnected modular network of individual decision points (i.e., pathway states). The clinical meta-pathway network provides a comprehensive framework for a plurality of diagnoses with a similar chief complaint instead of being limited to few very closely related diagnoses. The interactive clinical meta-pathway may be dynamically customized by a learning model to be utilized as a patient-specific clinical meta-pathway for improved accuracy and efficiency. The modular approach of using pathway states facilitates modification of certain pathway states without affecting other pathway states, or the whole, clinical meta-pathway network. In an embodiment, every pathway state contains a set of patient parameters (i.e., health variables) that are used to make a decision at each decision point within the pathway. The pathway state interconnects are determined based on analyses of health variables in light of the potential diagnoses at each pathway state. The disclosed embodiments provide a network of distinct diseases that are often separately analyzed for increased diagnosis space, accuracy, and clinical efficiency to directly benefit the healthcare process for physicians, patients, and more. Moreover, the disclosed embodiments utilizing the learning model may continuously optimize the clinical meta-pathway for effective representation of different groups of patients outside the “average patient” group as defined in evidence-based pathways.

The embodiments disclosed herein provide advantageous versatility in the clinical meta-pathway graph though the modular structure created from individual pathway states. Not only is the generated clinical meta-pathway comprehensive to better represent the complexity of the human body, but also granular by representing portions of the diagnosis space separated into decision points. In order to keep up to date with the quickly developing clinical research and the particularities in specific environments, clinical meta-pathways that are easily adaptable are highly desired. To this end, in an embodiment, separately modifiable pathway states are generated to accept new information, such as clinical research data, community data, health facility data, individual patient data, and more. Such new information, in the embodiment, may be applied and incorporated appropriately in the pathway states for enhanced accuracy and efficiency.

As an example, the overall clinical meta-pathway can be adapted to specific conditions by modifying specific variables to exclude certain equipment unavailable in a facility or at a certain time. Without recreating the whole, complex clinical meta-pathway graph, the pathway may be optimized for the facility at a certain time to maximize resources, in addition to increased accuracy and efficiency. Furthermore, continuous optimization and modification within each of the pathway states can be performed through a coupled learning engine based on, for example, artificial intelligence. It should be appreciated that such accurate and adapted clinical meta-pathway graphs may be utilized more frequently to increase accessibility and provide real-time assistance within the healthcare process. The modular structure of the clinical meta-pathway graph combines common pathway states to eliminate redundant collection and processing of data. In addition, the granular optimization enables partial modifications without reconstructing the overall clinical meta-pathway. To this end, it should be further appreciated that the clinical meta-pathway disclosed herein provides computer efficiency by reducing processing time, memory, and resources to implement such updates and to utilize the comprehensive clinical meta-pathways.

The embodiments disclosed herein also enable objective decision making in constructing and navigating through the clinical meta-pathway graph. Each pathway state includes a set of rules as functions (e.g., probabilistic functions, Boolean rules, etc.) that may be utilized to determine the connecting pathway states during construction. As noted above, the connection may also be modified for specific facilities, new research data, and the like, and any combination thereof, based on such set of rules. Moreover, navigating through the clinical meta-pathway, particularly in interactive navigation, can ultimately allow objective identification of at least one diagnosis, based on health variables, input data, and more, to increase accuracy and consistency through each decision point in the process and further in the final decision. Current clinical pathways provide some guidance through siloed evidence-based diagnosis, however, restricted in a breadth of diagnoses and execution in actual clinical settings. The clinical pathways in their current form are distributed in, for example, portable document format (PDF) through emails, web portals, applications, and the like, which provide predetermined pathways without allowing facilitated updates nor interactive guidance through the clinical pathway. That is, the rigid nature of the current clinical pathways reduces accessibility and availability in actual clinical practices. To this end, though clinical pathways are created to benefit, they can be underutilized by physicians, for example, to rely on oneself to make subjective decisions through the diagnosis process and result in erroneous decisions and undesired variations in patient outcomes.

However, the clinical meta-pathway, in the disclosed embodiments, provides a computable framework including decision making standards defined by, for example, weights, scores, raking, and the like, of variables, to objectively analyze the input data of the patient by a user, such as a physician. In an embodiment, the objective decisions may guide the user of the interactive clinical meta-pathway through a faster and safer route and further enable the identification of less common diseases associated with the chief complaint that are not well known and otherwise difficult to identify through subjective decision making. It should be appreciated that the embodiments disclosed herein provide dynamic, interactive clinical meta-pathways that not only provides accurate and effective representation of the expanded clinical network, but also increases accuracy and consistency of decisions in utilizing the constructed clinical meta-pathways. It should also be appreciated that the system can generate automatic physician notes based on the progress of the pathway (e.g., the patient journey) that captures the navigation through the clinical meta-pathway.

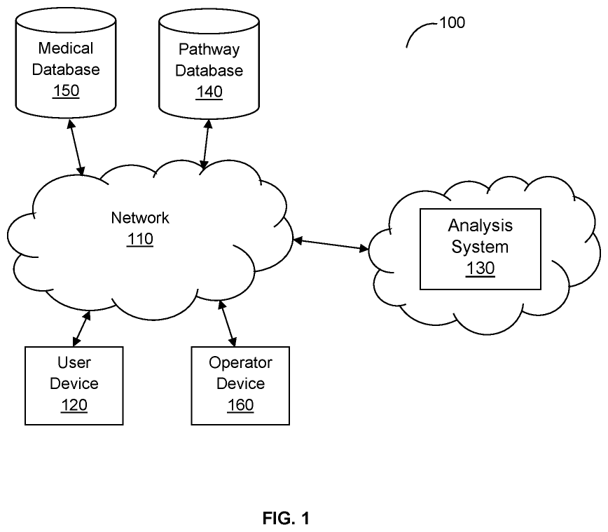

FIG. 1 shows an example network diagram 100 utilized to describe the various disclosed embodiments. In the example network diagram 100, a user device 120, an analysis system 130, a pathway database 140, a medical database 150, and an optional operator device 160 are communicatively connected via a network 110. The network 110 may be, but is not limited to, a wireless, cellular or wired network, a local area network (LAN), a wide area network (WAN), a metro area network (MAN), the Internet, the worldwide web (WWW), similar networks, and any combination thereof. The analysis system 130 may be deployed in a cloud computing platform which may be, but is not limited to, a public cloud, a private cloud, or a hybrid cloud. In some embodiments, the analysis system 130, when installed in the cloud, may operate as a software as a service (SaaS) and integrated to an electronic health record application programming interface (EHR API).

The user device (UD) 120 and the operator device 160 may each be, but is not limited to, a personal computer, a laptop, a tablet computer, a smartphone, a wearable computing device, or any other device capable of receiving, processing, and displaying notifications. The user device 120 may be associated with a medical entity, such as a physician, that can be presented with and/or interact with the clinical meta-pathway through a graphical user interface (GUI) presented on the user device 120. In addition, the user device 120 may be utilized for authorized users to modify the clinical meta-pathway based on the analyses from the analysis system 130. The optional operator device 160 may be associated with an operational entity, such as a billing department within the medical facility or external hospital, to provide streamline services and communications to patients.

In an embodiment, the user device 120 may be utilized to present alternative pathway states in the clinical meta-pathway enabling physician involvement (e.g., by selecting) through the navigation process. Here, the suggested pathway states, such as exams, tests, decisions, and the like, may be personalized for a specific patient and also for specific geographical areas or medical facilities, based on extracted and/or input data, such as but not limited to, patient data, facility data, and the like, and further by data from the analysis system 130. In another embodiment, the user device 120 may be utilized to input patient data such as, but not limited to, age, sex, ethnicity, chief complaint, test results, symptoms, and the like, through the graphical user interface for personalized execution of the clinical meta-pathway. In such a scenario, input patient data from the electronic health record application programming interface (EHR API) or the graphical user interface may be incorporated in the clinical meta-pathway as health variables for navigating through the pathway states (or decision points) and leading to a diagnosis.

The pathway database 140 may be part of the analysis system 130 or may be separate and communicatively connected via the network 110. The pathway database 140 may be utilized for storing, for example, disease profiles, respective health variables, pathway states, module profiles, and more, as well as any combination thereof. In addition, the database 140 may include medical research information from, for example, medical literature, academic papers, medical portals, and more. In an embodiment, the pathway database 140 may provide medical research information to the analysis system 130 for development of the clinical meta-pathway graph. In a further embodiment, the pathway database 140 may store progress data collected from executing the clinical meta-pathway. In an example embodiment, the progress data includes data collected along a patient's journey throughout the pathway. Such progress data may be utilized for analysis in at least one of a single pathway and aggregated meta-pathways. In an embodiment, the progress data is used as historical data to train the AI model and update the clinical meta-pathway graph.

The medical database 150 may communicate with the analysis system 130, either directly or over the network 110. The medical database 150 may be associated with the medical entity to store various medical information such as, but not limited to, patient electronic health records (EHR), facility information, equipment information, community data, and the like.

The analysis system 130 may be configured to collect medical research information and standardize the parameters including but not limited to, diagnosis, symptoms, tests, and more, to generate a plurality of disease profiles and an encompassing diagnosis space related to targeted chief complaints. It should be noted that the chief complaint may be associated with multiple diseases that are not typically categorized in a single clinical pathway protocol in conventional siloed protocols. The analysis system 130 may retrieve input data from databases, such as the pathway database 140 and the medical database 150, as well as user devices (e.g., user device 120 and operator device 160), to construct and update the disease profiles, and further modify the clinical meta-pathway graph.

In an embodiment, the analysis system 130 is configured to construct the clinical meta-pathway graph based on the plurality of disease profiles and parameters within the diagnosis space. Each clinical meta-pathway graph is associated with a chief complaint that may be branched out in an interconnected network of pathway states. The pathway states may be connected to one or more other pathway states to ultimately lead onto an endpoint state to conclude at least one path within the clinical meta-pathway graph. In an embodiment, the endpoint state may include at least one diagnosis associated with the chief complaint of the clinical meta-pathway graph. The clinical meta-pathway graph created herein within may provide recommended paths for analyzing various input data.

In an embodiment, during the construction phase of the clinical meta-pathway, at least one algorithm may be applied to parameters in the diagnosis space to generate a plurality of pathway states, which are further connected to create a comprehensive clinical meta-pathway graph. In further embodiment, during the implementation phase, the analysis system 130 may be configured with an artificial intelligence (AI) engine (not shown) that can apply at least one algorithm, such as a machine learning algorithm, on real-time input data, for example, patient data, physician selection, community data, historical data, and more, to dynamically provide accurate recommendations for customized and optimized clinical meta-pathway implementation. It should be noted that, in the implementation phase, such input data are utilized to navigate through the customized clinical meta-pathway.

In an embodiment, the AI engine may be configured to dynamically create and train an AI model from input data, such as but not limited to, historical patient data, real-time data, and more, in order to customize the constructed clinical meta-pathway graph for the specific patient. In an embodiment, the AI engine may calculate the resolution function (or module function) of a specific pathway state in the clinical meta-pathway with respect to particular input data to efficiently and accurately guide navigation through the clinical meta-pathway during implementation. In another embodiment, the AI engine may be configured to optimize the structure of the clinical meta-pathway graph and present to authorized medical personnel via a user device 120, for example, for auditing and reviewing. It should be noted that the modular structure of the clinical meta-pathway graph including multiple, but individual, pathway states facilitate editing and modification of certain components or portions of the clinical meta-pathway graph without complete recreation of the whole clinical meta-pathway graph.

According to the disclosed embodiments, the AI engine is configured to customize the clinical meta-pathway that represents the diagnosis space based on a physiological knowledge of a patient and/or patient group. Such clinical meta-pathway may be used to assess the a priori probabilities of various physiological conditions such as certain diseases given a partial set of patient parameters as are observed at the beginning of a medical treatment. In addition, such a clinical meta-pathway can be used to assess the dynamic differential probability of those conditions upon arrival of new patient information throughout the pathway implementation. It should be noted that changes determined by the AI engine can be readily incorporated into the flexible framework of the clinical meta-pathway for improvements in specificity for patients.

It should be noted that the clinical meta-pathway provides quantification at each and every decision point (i.e., pathway states) enabling objective decision making. Moreover, the disclosed embodiments and the clinical meta-pathway described herein continuously improves rules (or criteria) for decisions at each pathway state that are tailored to specific patient and/or patient groups for improved accuracy. It should be further noted that customization of the clinical meta-pathway allows navigation through selective pathway states minimizing repeated analysis to not only improve accuracy but also improve computer efficiencies by conserving processing power and memory.

Moreover, the analysis system 130 may be configured to allow navigation through the constructed clinical meta-pathway graph. In an example embodiment, the analysis system 130 may retrieve first input data, such as but not limited to, patient data (e.g., from EHR), facility data, and the like, to be implemented within the diagnosis space of the clinical meta-pathway graph associated with the chief complaint. Such first input data and second input data, such as but not limited to physician input data, operator input data, and the like, together may be utilized for objective decision making at the pathway states and navigating through the clinical meta-pathway graph. The first and second input data may be used to update variables, associated values, resolution functions, and the like, within the clinical meta-pathway state. The next pathway states for each of the pathway states may be selected based on the constructed clinical meta-pathway graph and updated variables. And thus, depending on the selection of the next pathway state, the navigation path within the clinical meta-pathway graph may differ. In an embodiment, navigation through the clinical meta-pathway graph may enable accurate analysis and identification of clinical actions for a particular patient, potentially identifying at least one diagnosis probable for the chief complaint based on the first and second input data. In an embodiment, second input data may be received through the user devices (e.g., user device 120 and operator device 160) over the network and may be stored in, for example, the memory of the analysis system 130, pathway database 140, and any combination thereof.

According to the disclosed embodiments, navigation through the clinical meta-pathway may be recorded as progress data and utilized to generate a smart log via a GUI and/or automatic physician notes. The progress data may include each pathway state taken through the clinical meta-pathway graph and suggestions provided to a user at each pathway state. In an example embodiment, the generated physician note may be presented as part of a patient journey report for display at a user device. In another example embodiment, the generated physician notes are directly accessible from authorized personnel via the operator device 160 for analysis. In yet another example embodiment, the generated physician notes may be exported to the EHR and stored at, for example, the medical database 150. In an embodiment, the generated physician notes may be stored at a pathway database 140. As an example, a person in the billing department can access the physician note describing progress through the clinical meta-pathway to accurately and efficiently determine billing information in association to actual actions taken.

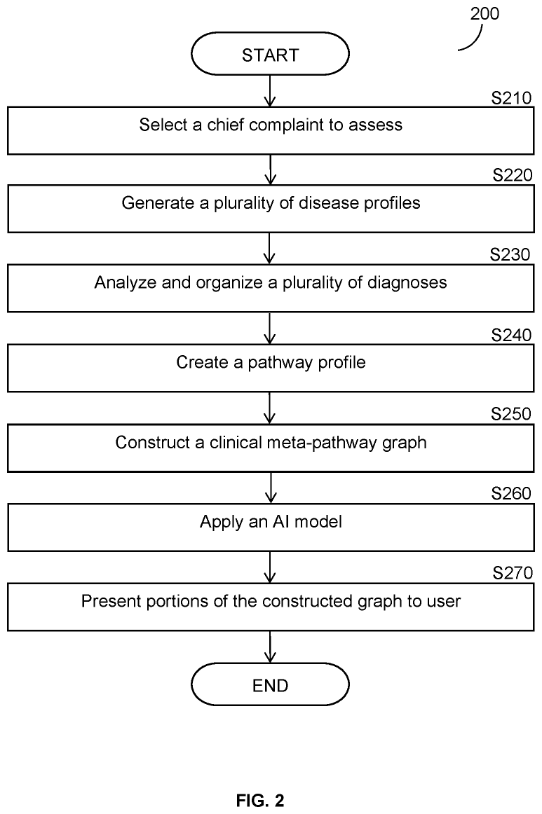

FIG. 2 is an example flowchart 200 illustrating a method for constructing an interactive clinical meta-pathway according to an embodiment. The method described herein may be executed by the analysis system 130, FIG. 1.

At S210, a chief complaint to assess is selected. The chief complaint of concern is a common symptom that is related to a large pool of diagnosis, for example and not limited to, chest pain, back pain, dizziness, and more. In an embodiment, a differential diagnosis (DD) set may be created including a plurality of diagnoses (or diseases) that are associated with the chief complaint (or use-case). As an example, a DD set of the chief complaint, chest pain, may include more than 50 different potential diagnoses. In an embodiment, a separate interactive clinical meta-pathway may be created for each of the different chief complaints.

At S220, a plurality of disease profiles is generated for each diagnosis in the differential diagnosis (DD) set. Each disease profile includes elements associated with the disease that are standardized and referred to as health variables. In an embodiment, the health variables may be, for example, risk factors, symptoms, tests, measurements of physiological condition of the patient, medications, treatments, genetic factors, and more. The health variable may include information such as, but not limited to, type of variable, values for each of the variables, relation of variable to disease (e.g., factor or symptom), likelihood of variable relative to disease, and the like, in a canonical format. In an embodiment, the disease profiles are initially created based on evidence-based data (or first input data) such as, but not limited to, medical research information, standardized parameters, and the like received from the pathway database (e.g., the pathway database 140, FIG. 1).

As an example, a disease profile for diabetes may include excessive fatigue, high blood sugar level, blood test, family history, insulin therapy, and more. In an embodiment, health variables may be added to disease profiles even after generation of the clinical meta-pathway to expand further to include, for example, medications, treatments, genetic factors, physiological states, and co-dependencies between diseases. In a further embodiment, the health variables and values associated within may be updated with utilization of the interactive clinical meta-pathway to provide additional input data for example, but not limited to, patient data, facility data, and the like, and applying algorithms, such as, machine learning algorithms. In an embodiment, the plurality of disease profiles may be stored in a memory of the analysis system 130, FIG. 1 and/or the pathway database 140, FIG. 1.

At S230, the plurality of diagnoses of the DD set is analyzed and organized. In an embodiment, the plurality of diagnoses may be organized in a risk assessment table including one or more clusters of at least a portion of the plurality of diagnoses that are grouped according to their respective risk scores. The risk scores for each of the diagnoses may be determined by applying at least one algorithm to the respective health variables. In an example embodiment, the diagnoses may be grouped into risk groups (or risk levels) of critical, urgent, and non-urgent, which may be utilized to prioritize a certain risk group over another. In a further embodiment, the plurality of diagnoses may be organized in an organ-system table that includes one or more system groups, for example, a central nervous system, a respiratory system, a cardiovascular system, and the like, of at least a portion of the plurality of diagnoses of the respective organ system. The organ system may be determined based on health variables associated with each of the diagnoses.

In one embodiment, a significant variable list including at least one health variable that indicates the related diagnosis may be determined for each of the plurality of diagnoses. A cost for obtaining values for each significant variable may be determined and represented using, for example, a numeral value from 0 to 1, where 0 indicates that the significant variable can be obtained relatively easily from a patient or a patient's EHR, and 1 indicates that such significant variable is relatively difficult to obtain, for example, an examination that uses a specialized equipment or results in a high bill. In another example embodiment, the costs of the significant variable may be represented using integers where, similarly, 0 indicates relatively easily obtainable variables and the difficulty to achieve values for the variables increase with increase in number. In an embodiment, the significant variable list is created from the health variables included in the disease profile for each diagnosis in the DD set.

In an embodiment, one or more resources that are used to obtain values for the significant variables of costs greater than 0 may be collected to create a resource table. The resource table includes a list of external resources used to determine values of the significant variables, other than patient data, EHR, or the like. The resource table may include, without limitation, blood testing, genetic testing, physical testing, imaging techniques, equipment, and more. In an embodiment, the contents of the resource table may be modified depending on the availability of resources.

In another embodiment, a variable comparison table including all the plurality of diagnoses in the DD set and the respective significant variables as determined in the significant variable lists may be created. Moreover, a likelihood score, indicating preliminary relevance, of each significant variable with respect to a diagnosis may be indicated per diagnosis in the DD set. In an example embodiment, the variable comparison table may be organized to list each diagnosis of the DD set as columns and variables from the significant variable list as row. In a further example embodiment, the significant variables may be arranged numerically from low cost to high cost and the plurality of diagnoses may be arranged according to the risk assessment table from high to low risk.

At S240, a pathway profile is created. The pathway profile may include at least a portion of elements associated with the chief complaint of concern including, but not limited to, the generated plurality of disease profiles, the risk assessment table, the organ-system table, risk scores, significant variable lists, the variable comparison table, and more. In an embodiment, the collective set of data in the created pathway profile may define the diagnostic space of the interactive clinical meta-pathway. Different portions of the pathway profile may be used to construct the interactive clinical meta-pathway for the selected chief complaint. It should be noted that all elements in the pathway profile may be represented in a standardized format (or canonical representation) for applying at least one algorithm for computation.

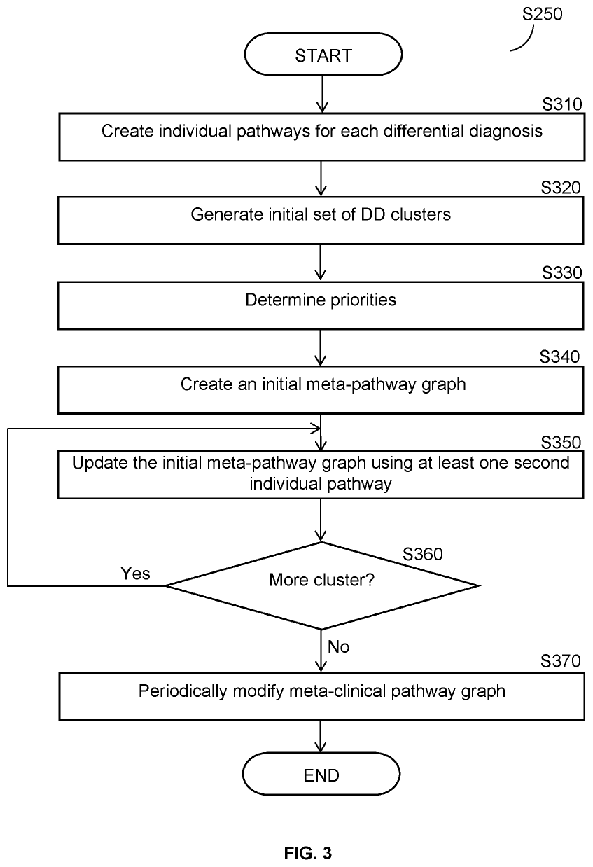

At S250, a clinical meta-pathway graph is constructed. The graph is constructed iteratively by generating and connecting a plurality of pathway states based on data included in the pathway profile. The pathway states are decision points that are connected to depict various paths that may be taken through the clinical meta-pathway graph. In an embodiment, individual pathways are created from evidence-based data that refer to the diagnostic process of one differential diagnosis or a small subset of adjacent differential diagnosis that are relevant to the chief complaint. In a further embodiment, the individual pathways are successively interweaved to one another to create a clinical meta-pathway graph of a chief complaint. Pathway states of individual pathways are extracted and/or combined to create an interconnected network of pathway states in the meta-pathway graph. The construction of the clinical meta-pathway graph is described in further detail below (FIG. 3).

In an embodiment, the clinical meta-pathway provides a comprehensive network of clinical pathways that integrate a wide range of potential diagnoses in a single clinical meta-pathway. In an embodiment, navigation through the clinical meta-pathway graph allows objective determination of clinical decisions and/or actions and potentially lead to at least one diagnosis associated with the chief complaint. In a further embodiment, the interweaving and interconnecting of a plurality of individual pathways eliminates similar pathway states (e.g., requesting input data, tests, exams, and the like) to reduce processing speed and conserve computer power and memory. It should be appreciated that the clinical meta-pathway graph provides a visual representation of the complex interconnects of diagnoses that may better reflect the human body than currently implemented clinical pathways that provide isolated pathways that often only represent single diagnosis. It should be further appreciated that such extension of the overall graph supports all related diagnoses while optimizing navigation efficiency through the diagnoses space.

In an embodiment, the generated plurality of pathway states that compose the clinical meta-pathway graph may be connected to one or more other pathway states. In an embodiment, a module profile may be created for each of the pathway states to include, for example but not limited to, a set of module DD set, a set of module variables resolution function, a list of next steps (i.e., next pathway states connected to current pathway state), and the like. Elements in the module profile may all be associated with the potential diagnoses applicable and included in the set of module DD set. The set of module variables includes health variables associated with the pathway state. As an example, a pathway state for a blood test includes hemoglobin concentration, cholesterol level, and more. In an example embodiment, the set of module variables may be identified from the variable comparison table and include respective costs for the module variables. In a further embodiment, the pathway state may include an outstanding module variable from the set of module variables (e.g., hemoglobin concentration) that is used to determine the next step from the respective pathway state.

The resolution function is a distribution function over a set of module variables that may be utilized to determine a relative probability of the next pathway state connected to the current pathway state. The list of next steps may include a plurality of next pathway states that are connected to the current pathway state. In an embodiment, the next pathway state may be determined by computing the resolution functions, as a set of rules, using the module variables. The next pathway state may be determined in order to rule out and/or rule in at least one diagnosis in the module DD set. In an embodiment, the pathway state may be an assessment state that helps resolve the diagnosis most effectively. For example, the assessment state may request a blood iron level reading from the patient's EHR in order to identify and determine the next steps of the pathway state.

In further embodiment, the assessment state may include a “to-do” action such as, but not limited to, a test or examination, an ultrasound imaging, and the like, to be performed and to retrieve one or more values for at least one of the module variables. In an example embodiment, the assessment state may be preconditioned to order the “to-do” action to be performed immediately for direct implementation when executing the clinical meta-pathway. In a further embodiment, the pathway state may be a decision state (or an endpoint state) that completes the current clinical pathway process and ends the pathway. It should be noted that a pathway may reach a decision point without reaching a definite diagnosis.

At S260, an AI model is applied to the constructed clinical meta-pathway graph. The AI model is created and trained using historical patient data for implementation of the clinical meta-pathway (i.e., during navigation through the clinical meta-pathway graph). In an embodiment, the AI model may be dynamically trained and updated by historical data from a specific patient to create a patient-specific AI model. The patient-specific AI model may be utilized to determine values for health variables and select certain variables over others to create a patient-specific clinical meta-pathway graph. In addition, resolution functions of pathway states may be updated for the patient-specific clinical meta-pathway graph from the training phase. That is, navigation through the constructed clinical meta-pathway may be customized for the specific patient to provide efficient and accurate clinical operations. In an embodiment, the customized clinical meta-pathway is navigated using input patient data and directed by the determined values and resolution functions. It should be noted that the customized clinical meta-pathway graph includes relevant pathway states for the patient or group of patients and excludes collecting and processing of irrelevant data with respect to the patient, diagnoses, and the chief complaint. In an embodiment, the AI model may be initially created based on evidence-based medical knowledge.

As an example, based on the trained AI model, the clinical meta-pathway of a diabetes patient may be led to one cluster of the DD set over another cluster or may be directed to avoid a certain “to-do” action that can be unclear for effective rule in or rule out of the diagnoses. In an embodiment, the variables may be re-arranged and/or resolution functions may be dynamically updated within the pathway states as an implementation of the AI model. Such editing of portions of the clinical meta-pathway may enable fine tuning of variables and scores to increase accuracy of the generated clinical meta-pathway and results obtained through utilization for the specific patient. Moreover, it should be appreciated that the clinical meta-pathway may be modified readily and rapidly focused on a small portion of the meta-pathway, due to the modular nature, without reconstructing all parts of the meta-pathway. In an embodiment, the historical patient data may be retrieved from a database (e.g., the medical database 150, FIG. 1). In further embodiments, the AI model may be generated and trained by the AI engine (not shown) in the analysis system (e.g., the analysis system 130).

At S270, portions of the clinical meta-pathway graph are presented to a user via a user device (e.g., the user device 120, FIG. 1). In an embodiment, the user may be a physician navigating through the clinical meta-pathway graph to determine at least one diagnosis of a patient. In an embodiment, portions of the clinical meta-pathway graph may be presented on a user device through a graphical user interface (GUI). In an example embodiment, portions of the clinical meta-pathway graph include current pathway state and list of next pathway states during navigation through the clinical meta-pathway, health variables, related diagnoses, and the like, of the pathway state. In a further embodiment, the portions presented may include recommended next pathway states based on the patient-specific clinical meta-pathway graph generated through implementation of the AI model. In an embodiment, the recommendation is performed dynamically and in real-time as patient data, such as but not limited to, historical data, EHR data, and the like, for the current patient are received.

The GUI may be used to input selections on at least one pathway state from the DD set of the respective pathway state. In another embodiment, the GUI may be used to provide certain input data including, but not limited to, demographics, test results, allergies, prior conditions, symptoms, and the like, when such input data is not available through the EHR stored in the medical database (e.g., medical database 150, FIG. 1). In another embodiment, the user may be an authorized user allowed to audit the clinical meta-pathway graph and, when appropriate, provide feedback and/or modify portions of the pathway profile of the clinical meta-pathway graph. It should be appreciated that the method illustrated herein within may be performed for various chief complaints to generate multiple clinical meta-pathways.

FIG. 3 is an example flowchart S250 illustrating a method for constructing a clinical meta-pathway graph by generating pathway states according to one embodiment. As noted above, the clinical meta-pathway graph is constructed by iteratively generating a plurality of individual pathways, each for a differential diagnosis and connecting existing and newly generated pathway states based on the plurality of individual pathways. The method described herein may be executed by the analysis system 130, FIG. 1.

At S310, individual pathways are created for each differential diagnosis. The individual pathways each includes a plurality of pathway states for a single differential diagnosis within the DD set. Each individual pathway is created based on health variables associated with the respective differential diagnosis including, for example, but not limited to, risk factors, symptoms, tests, measurements of physiological conditions of the patient, medications, treatments, genetic factors, and the like, and more. Here, risk factors represent patient parameters that can increase risk for physiological conditions such as, but not limited to age, medical history, and the like, and symptoms represent physiological expressions of such conditions, for example and without limitation, blood pressure, blood test results, and the like. The construction utilizes the evidence-based data of the differential diagnosis that are extracted from known diagnostic protocols. In an embodiment, the pathway states are connected to one another based on, for example, evidence-based data (e.g., medical research information, etc.) collected from a database (e.g., the pathway database 140, FIG. 1). In some embodiments, the created individual pathways are displayed at a user device via a graphical user interface for feedback and/or modification by the user (e.g., a physician).

At S320, an initial set of DD clusters is generated. The DD clusters may be based on the clusters of diagnoses as organized in the risk assessment table by risk scores (e.g., urgency) and/or the organ-system table by associated organ systems. As an example, based on the risk assessment table, one cluster includes DDs that have high morbidity, and another cluster includes DDs with mild side effects. In another example, based on the organ-system table, one cluster includes differential diagnoses (DDs) that occur in the heart and another cluster includes DDs that are detected in the brain.

At S330, priorities are determined between the DD clusters and the DDs within each cluster. The priorities are utilized to list DD clusters based on high priority to low priority which may be defined by at least one of the risk scores and the organ-system. As an example, a DD cluster associated with the heart has higher priority over a DD cluster relating to the kidney. In an embodiment, the DDs within each DD cluster are ranked from high priority to low priority. The priorities between the DDs within the DD cluster are determined based on, for example, respective risk scores. In an embodiment, the priorities may be defined by a plurality of rules that are determined based on at least one algorithm. In another embodiment, the plurality of rules may be predetermined. In some embodiments, the DDs within the cluster are randomly ordered.

At S340, an initial meta-pathway graph is created based on a first individual pathway. The initial meta-pathway graph mirrors the first individual pathway including pathway states, health variables, resolution function, list of next steps, associated differential diagnoses, and more. In an example embodiment, the first individual pathway is the first DD of the first DD cluster as determined from priorities (S330). In another example embodiment, the first individual pathway is of the DD with the highest priority between all DDs associated with the chief complaint.

At S350, the meta-pathway graph is updated based on at least one second individual pathway. The second individual pathways are the any individual pathways, other than the first individual pathway, created for each of the DDs within the respective DD cluster. In an embodiment, the updating of the meta-pathway graph is performed sequentially by applying one second individual pathway at a time. As an example, the initial meta-pathway graph is updated using the individual pathway of the DD that is second in line after the first individual pathway. In the same example, such updated meta-pathway graph is further updated using the individual pathway of the DD that is third in line after the first and second individual pathways, and so on until all DDs within the respective clusters are applied to update the meta-pathway graph.

In an embodiment, the at least one second individual pathway within the respective DD cluster is overlaid on the initial meta-pathway graph. In an embodiment, common pathway states between the first individual pathway and the at least one second individual pathway are identified and used to overlay the individual pathways. The common pathway states are similar pathway states indicating, for example, similar preliminary physician evaluation, specific blood test, specific radiology test, and the like. Health variables associated with the common pathway state may be combined and/or new health variables may be determined for the common pathway state collected from individual pathways. Similarly, the resolution functions of the common pathway state may be updated by combining or re-calculating resolution functions of the common pathway state in each individual pathway. In an embodiment, resolution functions may be generated based on health variables to determine the next state.

In an embodiment, next pathway states connecting to the common pathway states are determined, which may include at least one next pathway state that was connected to the common pathway state in the individual pathways. As an example, the common pathway state includes a test for blood iron level and the next pathway states includes a pregnancy test, dietary study, and coloscopy. In the same example, the meta-pathway graph is updated to includes one or more of the next pathway states in connection to the common pathway state to test for blood iron level.

In an example embodiment, an existing assessment state to perform the “to-do” action may be selected as the next pathway state. In another embodiment, when the assessment state for the “to-do” action is absent, a new assessment state may be created and determined as the next pathway state. In an embodiment, the determined next pathway state is connected to the pathway state as the subsequent step in the clinical meta-pathway graph. In an embodiment, the pathway state may have a list of next steps that includes at least one next pathway state that is connected as the successive pathway state in a path of the clinical meta-pathway graph. In an example embodiment, the next pathway state may be an endpoint pathway state including at least one diagnosis.

At S360, a check is performed whether there are more DD clusters. If so, operation continues to S350, otherwise, operation continues to S370. Operation at S350 is performed to continuously update the meta-pathway graph using DDs of the next DD cluster.

In an embodiment, the DDs of the next DD cluster are sequentially used to update the meta-pathway graph. The operation of S350 may be performed until no unresolved DD cluster remains out of the initial set of DD clusters that were generated. Here, the update using the next individual pathway is performed on the updated meta-pathway graph from the preceding individual pathway. For example, an individual pathway that is first in line in the next DD cluster is applied on the updated meta-pathway graph from applying all individual pathways in the previous DD cluster. In an embodiment, a complete clinical meta-pathway graph for the selected chief complaint may be achieved upon repeating and resolving all the DD clusters. It should be further noted that the updated meta-pathway graph is a network of pathways connecting pathway states of many differential diagnoses associated with the chief complaint to provide a comprehensive understanding of the diagnosis space. In addition, such a complex network enables discovery of new diagnoses, relations, tests, and the like, that are otherwise not revealed.

At S370, the clinical meta-pathway graph is periodically modified. Portions of the clinical meta-pathway graph may be modified with new medical information. In an example embodiment, a previously unrelated differential diagnosis may be discovered to be associated with the chief complaint. Pathway states are created for the differential diagnosis to update the clinical meta-pathway. In an embodiment, the meta-pathway graph is updated based on implementation (e.g., navigation through the clinical meta-pathway) and feedback received, for example, from a user device.

In an embodiment, the constructed clinical meta-pathway graph may be optimized by modifying portions of the clinical meta-pathway. The modular nature of the clinical meta-pathway facilitates modification of, for example, elements of certain pathway states, without disturbing the whole clinical meta-pathway graph. It should be appreciated that such modular structure and modifications enabled thereof reduces processing time and power for optimization. As an example, parts of the clinical meta-pathway graph may be statically modified for a unique medical facility in order to accurately represent the available resources in the facility.

It should be noted that the method of FIG. 3 is depicted for illustrative purposes and does not limit the scope of the disclosed embodiments.

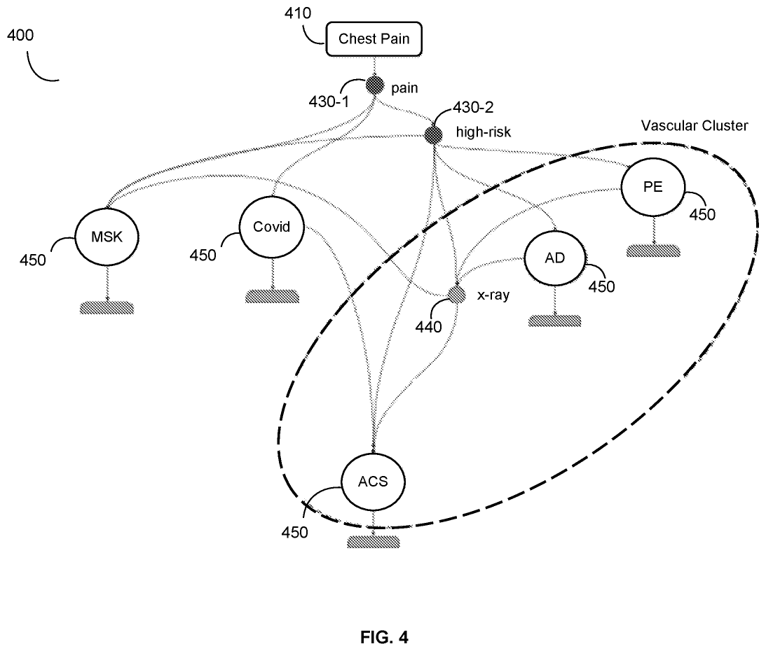

FIG. 4 is an example schematic diagram of a simplified clinical meta-pathway graph 400 according to an example embodiment. The clinical meta-pathway graph 400 is constructed for a chief complaint 410 of chest pain, as an example, to include a plurality of potential diagnoses, musculoskeletal (MSK), COVID-19, autonomic dysreflexia (AD), pulmonary embolism (PE), acute coronary syndrome (ACS), in the differential diagnosis (DD) set. The clinical meta-pathway graph 400 includes multiple pathway states 430 through 450 that may be categorized as at least an assessment state 430 (430-1 and 430-2), an operative state 440, and a decision state 450, that may individually act as decision points for navigating through the clinical meta-pathway graph 400. The assessment state 430, the operative state 440, and the decision state 450 may be simply referred to as pathway states within the clinical meta-pathway graph 400.

Each pathway state may be connected to one or more other pathway states, where the pathway state moving away from the chief complaint 410 may be the next pathway states of a respective pathway state. The complex network of pathway states in the clinical meta-pathway graph 400 may enable discovery of potential diagnosis within the DD set without excluding diagnosis that may be less commonly known. It should be noted that the potential diagnosis in the DD set may change at different pathway states. As an example, a “pain” assessment state 430-1 may include all five potential diagnoses in the DD set, whereas a “high-risk” assessment state 430-2 may only include four potential diagnoses in the DD set, excluding Covid.

In addition, the clinical meta-pathway graph 400 may indicate clusters of diagnoses according to organ-system, for example a vascular cluster of PE, AD, and ACS, and further according to the risk level, for example a high-risk cluster of MSK, ACS, AD, and PE, which branch out from the “high-risk” assessment state 430-2.

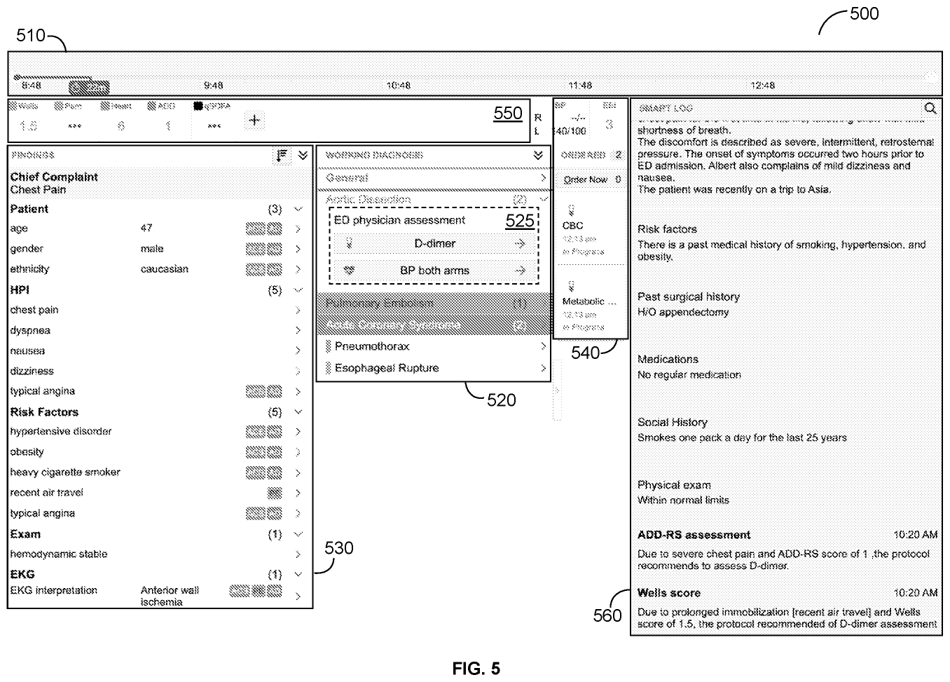

FIG. 5 is an example of a graphical user interface (GUI) 500 for interacting with the clinical meta-pathway according to an example embodiment. As disclosed above, a user device (e.g., user device 120, FIG. 1) may be configured to display portions of the clinical meta-pathway graph using the GUI 500. In an embodiment, a user may be presented with portions of the patient-specific clinical meta-pathway that include dynamically determined recommendations for the specific patient. Moreover, the GUI 500 may enable a user, such as a physician, to interact and navigate through the clinical meta-pathway for selecting path direction and ultimately, diagnosis of a patient.

In an embodiment, the GUI 500 may display a patient journey 510 that provides a progress map of the patient including a chief complaint and each pathway state taken within the clinical meta-pathway. In an example embodiment, the patient journey 510 may be shown as a timeline as shown in the patient journey box 510 from 8:48 to beyond 12:48. In addition, the differential diagnosis (DD) set 520 at the current step (i.e., pathway state) may be displayed based on the chief complaint, previous steps, and, in certain cases, further based on specific patient data. In an embodiment, a list of next steps 525 is shown within the DD set 520 to present recommended next steps (i.e., the next pathway state) that may potentially rule out certain DDs within the DD set 520. In the example embodiment, the DD set 520 includes aortic dissection, pulmonary embolism, and acute coronary syndrome and a next step of ED physician assessment to rule out (or rule in) aortic dissection as shown in respective boxes 520 and 525.

In an example embodiment, the GUI 500 may also show patient parameters 530 associated with the current step (i.e., the pathway state or decision point) and a list of pending tasks 540 from the previous steps of the pathway. The patient parameters 530 may list the chief complaint (e.g., chest pain) as well as patient-specific values to the health variables concerned in the current step and/or aggregated from previous steps. In the non-limiting example, the patient parameters 530 include basic patient information (e.g., age, gender, ethnicity), history of present illness (HPI) (e.g., chest pain, dyspnea, nausea, dizziness, and typical angina), risk factors (e.g., hypertensive disorder, obesity, heavy cigarette smoker, recent air travel, and typical angina), exam (e.g., hemodynamic stable), electrocardiogram (EKG) (e.g., EKG interpretation) and applicable values for each of these parameters.

The pending tasks 540 may list the “to-do” actions underway and performed from the previous steps. In an embodiment, the results of the pending tasks 540 may be utilized to update patient parameters 530 and/or to make the decision at the current step. In the non-limiting example, as shown in pending tasks box 540, the pending tasks include a complete blood count (CBC) and a metabolic blood test that are both in progress. Each of the plurality of diagnoses in the DD set 520 may be color coded with tags to correlate specific diseases to related patient parameters 530 of the current step and corresponding pending tasks 540. As an example, the box of patient parameters 530 includes a tag of acute coronary syndrome (ACS) and aortic dissection (AD) in the “age” parameter for the patient.

In addition, a plurality of scores 550 may be displayed to indicate risk levels based on evidence for the differential diagnoses associated with the current step. The example shown in the box for the plurality of scores 550 indicates a wells score of 1.5 and a heart score of 6. In an example embodiment, such scores may be utilized to rule out diagnoses from the DD set 520 in the pathway. In an example embodiment, a user of the GUI 500 may rule out certain diagnoses from the DD set based on the patient parameters 530 and results from the pending tasks 540, which will change the contents of the GUI 500 to present a new DD set 520 and associated patient parameters 530 for the new current step. It should be noted that the GUI 500 is utilized to present portions of the clinical meta-pathway without the actual analysis that are performed within the analysis system (e.g., the analysis system 130, FIG. 1). In addition, a smart log 560 may be presented to a user via the GUI. The smart log 560 provides suggested physician notes for every step taken in the patient journey, which can later be edited and translated into a text report to be sent back to the physician notes section in the EHR. The smart log 560 can be filtered and searched based on user input, and is updated automatically once pending tasks have been completed. In a non-limiting example, as shown in the smart log box 560, the smart log describes the following:
The discomfort is described as severe, intermittent, retrosternal pressure. The onset of symptoms occurred two hours prior to ED admission. Albert also complains of mild dizziness and nausea, The patient was recently on a trip to Asia.
Risk factors:
There is a past medical history of smoking, hypertension, and obesity.
Past surgical history:
H/O appendectomy
Medications:
No regular medication
Social History:
Smokes one pack a day for the last 25 years
Physical exam:
Within normal limits
ADD-RS assessment:
Due to severe chest pain and ADD-RS score of 1, the protocol recommends to assess D-dimer.
Wells score:
Due to prolonged immobilization [recent air travel] and Wells score of 1.5, the protocol recommended of D-dimer assessment

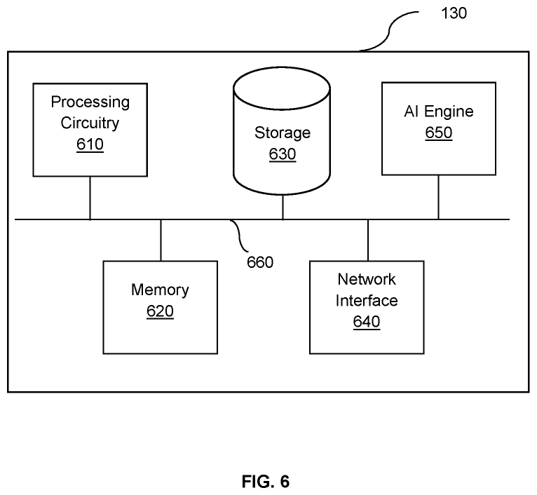

FIG. 6 is an example schematic diagram of an analysis system 130 according to an embodiment. The analysis system 130 includes a processing circuitry 610 coupled to a memory 620, a storage 630, a network interface 640, and an artificial intelligence (AI) engine 650. In an embodiment, the components of the analysis system 130 may be communicatively connected via a bus 660.

The processing circuitry 610 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 620 may be volatile (e.g., random access memory, etc.), non-volatile (e.g., read only memory, flash memory, etc.), or a combination thereof.

In one configuration, software for implementing one or more embodiments disclosed herein may be stored in the storage 630. In another configuration, the memory 620 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 610, cause the processing circuitry 610 to perform the various processes described herein.

The storage 630 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The network interface 640 allows the analysis system 130 to communicate with, for example, the network 110.

The AI engine 650 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI engine 650 is configured to perform, for example, machine learning based on input data such as patient data, selection data at pathway state, and more, received over the network 110.

It should be understood that the embodiments described herein are not limited to the specific architecture illustrated in FIG. 6, and other architectures may be equally used without departing from the scope of the disclosed embodiments.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for generating a patient-specific clinical meta-pathway, comprising:
constructing a clinical meta-pathway graph of a network of pathway states, wherein each pathway state is associated with at least one of a plurality of differential diagnoses of a chief complaint; and wherein each pathway state includes a set of rules that define a connecting pathway state;
applying a trained model to the constructed clinical meta-pathway graph to update the set of rules that define the connecting pathway state, wherein the model is trained based on at least historical patient data;
navigating through the clinical meta-pathway based on input patient data, wherein the navigation through pathway states is guided by a function of the set of rules; and
causing a display of at least a portion of the navigating pathway states.

2. The method of claim 1, further comprises:
generating, using first input data, a disease profile for each of the plurality of differential diagnoses of a chief complaint, wherein the disease profile includes health variables extracted from the first input data and associated to the differential diagnoses; and
determining the pathway states for the disease profile by organizing the health variables of the differential diagnosis.

3. The method of claim 1, wherein the pathway state is any one of: a decision state, an assessment state, and an endpoint state.

4. The method of claim 2, wherein the health variables are organized by at least one of: types, values, relations, and likelihood of the health variables.

5. The method of claim 2, wherein the first input data is at least one of: clinical research data, community data, health facility data, and patient data.

6. The method of claim 3, wherein the navigating further comprises:
determining a next pathway state of the assessment state by applying a set of rules on a test result from a “to-do” action.

7. The method of claim 1, further comprising:
generating a patient journey report describing the navigation through the clinical meta-pathway graph, wherein the patient journey report includes pathway states.

8. The method of claim 1, wherein constructing the clinical meta-pathway graph further comprises:
creating an initial meta-pathway graph based on a first individual pathway;
updating the initial meta-pathway graph by overlaying at least one second individual pathway to the initial meta-pathway graph using a common pathway state, wherein the common pathway state is in the first individual pathway and the at least one second individual pathway, wherein updating further comprises modifying health variables and resolution functions of the common pathway state and determining next pathway states; and
sequentially repeating the update of the meta-pathway graph until all the at least one second individual pathways are used.

9. The method of claim 8, further comprising:
creating individual pathways for the plurality of differential diagnoses, wherein each of the individual pathways is associated with one of the plurality of differential diagnoses;
generating an initial set of DD clusters based on risks and organ systems, wherein the DD clusters include subsets of the plurality of differential diagnoses;
determining priorities of DD clusters and the subset of the plurality of differential diagnoses within the DD clusters; and
identifying the at least one second individual pathway based on the determined priorities and the DD clusters.

10. A non-transitory computer readable medium having stored thereon instructions for causing a processing circuitry to execute a process, the process comprising:
constructing a clinical meta-pathway graph of a network of pathway states, wherein each pathway state is associated with at least one of a plurality of differential diagnoses of a chief complaint; and wherein each pathway state includes a set of rules that define a connecting pathway state;
applying a trained model to the constructed clinical meta-pathway graph to update the set of rules that define the connecting pathway state, wherein the model is trained based on at least historical patient data;
navigating through the clinical meta-pathway based on input patient data, wherein the navigation through pathway states is guided by a function of the set of rules; and
causing a display of at least a portion of the navigating pathway states.

11. A system for generating a patient-specific clinical meta-pathway, comprising:
a processing circuitry; and
a memory, the memory containing instructions that, when executed by the processing circuitry, configure the system to:
construct a clinical meta-pathway graph of a network of pathway states, wherein each pathway state is associated with at least one of a plurality of differential diagnoses of a chief complaint; and wherein each pathway state includes a set of rules that define a connecting pathway state;
apply a trained model to the constructed clinical meta-pathway graph to update the set of rules that define the connecting pathway state, wherein the model is trained based on at least historical patient data;
navigate through the clinical meta-pathway based on input patient data, wherein the navigation through pathway states is guided by a function of the set of rules; and
cause a display of at least a portion of the navigating pathway states.

12. The system of claim 11, wherein the system is further configured to:
generate, using first input data, a disease profile for each of the plurality of differential diagnoses of a chief complaint, wherein the disease profile includes health variables extracted from the first input data and associated to the differential diagnoses; and
determine the pathway states for the disease profile by organizing the health variables of the differential diagnosis.

13. The system of claim 11, wherein the pathway state is any one of: a decision state, an assessment state, and an endpoint state.

14. The system of claim 12, wherein the health variables are organized by at least one of: types, values, relations, and likelihood of the health variables.

15. The system of claim 12, wherein the first input data is at least one of: clinical research data, community data, health facility data, and patient data.

16. The system of claim 13, wherein the system is further configured to:
determine a next pathway state of the assessment state by applying a set of rules on a test result from a “to-do” action.

17. The system of claim 11, wherein the system is further configured to:
generate a patient journey report describing the navigation through the clinical meta-pathway graph, wherein the patient journey report includes pathway states.

18. The system of claim 11, wherein the system is further configured to:
create an initial meta-pathway graph based on a first individual pathway;
update the initial meta-pathway graph by overlaying at least one second individual pathway to the initial meta-pathway graph using a common pathway state, wherein the common pathway state is in the first individual pathway and the at least one second individual pathway, wherein updating further comprises modifying health variables and resolution functions of the common pathway state and determining next pathway states; and
sequentially repeat the update of the meta-pathway graph until all the at least one second individual pathways are used.

19. The system of claim 18, wherein the system is further configured to:
create individual pathways for the plurality of differential diagnoses, wherein each of the individual pathways is associated with one of the plurality of differential diagnoses;
generate an initial set of DD clusters based on risks and organ systems, wherein the DD clusters include subsets of the plurality of differential diagnoses;
determine priorities of DD clusters and the subset of the plurality of differential diagnoses within the DD clusters; and
identify the at least one second individual pathway based on the determined priorities and the DD clusters.
